SEQUENCE LISTING (10 sequences)

SEQ ID NO: 1 - protein, 5 aa, synthetic construct
NYWIG

SEQ ID NO: 2 - protein, 17 aa, synthetic construct
IIDPSNSYTRYSPSFQG

SEQ ID NO: 3 - protein, 8 aa, synthetic construct
WYYKPFDV

SEQ ID NO: 4 - protein, 14 aa, synthetic construct
TGSSSNIGSGYDVH

SEQ ID NO: 5 - protein, 7 aa, synthetic construct
GNSKRPS

SEQ ID NO: 6 - protein, 11 aa, synthetic construct
ASWTDGLSLVV

SEQ ID NO: 7 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 8 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 9 - protein, 447 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2025/0306035 A1
(43) Pub. Date: Oct. 2, 2025

(54) METHODS FOR PREDICTING TREATMENT RESPONSE IN PSORIASIS

(71) Applicant: Janssen Biotech, Inc., 19044, PA (US)

(72) Inventors: Yanqing Chen, San Diego, CA (US); Julianty Angsana, San Diego, CA (US); Brice Keyes, Poway, CA (US); Monica Leung, San Diego, CA (US)

(21) Appl. No.: 19/093,469

(22) Filed: Mar. 28, 2025

Related U.S. Application Data
(60) Provisional application No. 63/571,786, filed on Mar. 29, 2024.

(51) Int. Cl. G01N 33/68

(52) U.S. Cl. CPC G01N 33/6893; G01N 33/6863; G01N 2333/50; G01N 2333/54; G01N 2333/70596; G01N 2800/205; G01N 2800/52

(57) ABSTRACT

The disclosure provides a method of predicting a response to a treatment regimen for psoriasis in a subject. Biomarkers and clinical variables that can be used to predict the response and to select a treatment regimen are described herein. Also described is a kit for predicting a response to a treatment regimen for psoriasis in a subject.

### CROSS-REFERENCE TO RELATED APPLICATIONS

This application claims priority U.S. Provisional Patent Application No. 63/571,786, filed on Mar. 29, 2024, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an xml formatted sequence listing with a file name “JBI6896WOPCT1 Sequence Listing” and a creation date of Mar. 17, 2025, and having a size of 11 kb. The sequence listing submitted via USPTO Patent Center is part of the specification and is herein incorporated by reference in its entirety.

### FIELD OF THE DISCLOSURE

The present disclosure is directed generally to the detection or diagnosis of disease states, preferably psoriasis, to the identification of a treatment regimen for psoriasis, and/or to indicate the responsiveness to the treatment regimen for psoriasis in a subject, and provides methods, reagents, and kits useful for this purpose. Provided herein are a panel of biomarkers that are indicative of, diagnostic for and/or useful for identification of a treatment regimen, and/or are indicative of responsiveness to the treatment regimen for psoriasis, probes capable of detecting the panel of biomarkers and related methods and kits thereof.

### BACKGROUND

Psoriasis is a common, chronic immune-mediated skin disorder with significant co-morbidities, such as psoriatic arthritis (PsA), depression, cardiovascular disease, hypertension, obesity, diabetes, metabolic syndrome, and Crohn's disease. Plaque psoriasis is the most common form of the disease and manifests in well demarcated erythematous lesions topped with white silver scales. Plaques are pruritic, painful, often disfiguring and disabling, and a significant proportion of psoriatic patients have plaques on hands/nails face, feet and genitalia. As such, psoriasis negatively impacts health-related quality of life (HRQOL) to a significant extent, including imposing physical and psychosocial burdens that extend beyond the physical dermatological symptoms and interfere with everyday activities. For example, psoriasis negatively impacts familial, spousal, social, and work relationships, and is associated with a higher incidence of depression and increased suicidal tendencies.

Guselkumab (GUS) (also known as CNTO 1959) is a fully human IgG1 lambda monoclonal antibody that binds to the p19 subunit of IL-23 and inhibits the intracellular and downstream signaling of IL-23, required for terminal differentiation of T helper (Th)17 cells. Guselkumab is approved to treat moderate to severe plaque psoriasis, and psoriatic arthritis in adults.

GUIDE is an ongoing Phase III study that examines clinical and immunological impact of new treatment strategies with GUS in patients with moderate-to-severe plaque-type psoriasis. In GUIDE, subjects who achieved PASI=0 at both week (W) 20 and W28 were defined as super responders (SRe); all other subjects were labeled as non-SRe at W28. SRes with PASI<3 at W68 were withdrawn from treatment in part 3 of the study (W68-220). Subjects were monitored to see if they were able to maintain drug-free disease control (PASI≤5) following GUS withdrawal.

Currently, there are no identified blood-based biomarkers that allow for the prediction clinical response to treatment with GUS in psoriasis. Identification of markers that predict patients' clinical response to treatment and/or their ability to maintain drug-free disease control are of high value and will enable more tailored precision medicine approaches to treating psoriasis.

### SUMMARY OF THE INVENTION

In one general aspect, the disclosure relates to a method of predicting a response to a treatment regimen for psoriasis in a subject. The method can, for example, comprises:
a. obtaining a sample from the subject;
b. contacting the sample with a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A);
c. obtaining a panel of clinical variables from the subject comprising disease duration; body mass index (BMI), weight, age, sex, treatment history, Dermatology Life Quality Index (DLQI) score, and Psoriasis Area and Severity Index (PASI);
d. analyzing a range of threshold values of the panel of biomarkers and the panel of clinical variables to determine a predictive value for the subject, wherein a predictive value of less than about 0.1 indicates that the subject is less likely to respond to the treatment regimen than a subject with a predictive value greater than about 0.1;
e. determining the treatment duration for the subject with the treatment regimen based on the predictive value, with a score of greater than about 0.1 indicating treating the subject for a shorter duration and a score of less than about 0.1 indicating treating the subject for a longer duration; and
f. treating the subject with the treatment regimen for a duration based on the score.

In a specific embodiment, a predictive value of 0 indicates that the subject is less likely to respond to the treatment regimen than a subject with a predictive value of 1.

In certain embodiments, the contacting step comprises contacting the samples with an isolated set of probes corresponding to the panel of biomarkers. In a specific embodiment, the sample is a blood sample.

In certain embodiments, the method further comprises administering a therapeutic agent to the subject to treat or prevent the psoriasis. In certain embodiments, the therapeutic agent is an anti-IL-23 antibody. In a specific embodiment, the anti-IL-23 antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3.

In a specific embodiment, the anti-IL-23 antibody comprises a light chain variable region amino acid sequence of SEQ ID NO: 8 and a heavy chain variable region amino acid sequence of SEQ ID NO: 7. In a specific embodiment, the anti-IL-23 antibody comprises a light chain amino acid sequence of SEQ ID NO: 10 and a heavy chain amino acid sequence of SEQ ID NO: 9. In a specific embodiment, the anti-IL-23 antibody is guselkumab.

In certain embodiments, the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

In certain embodiments, the analyzing step is performed using a machine learning module. The machine learning model can, for example, be at least one of the following: a support vector machine module, a random forest module, a logistic regression module, or a gradient tree boosting module.

In certain embodiments, the shorter treatment duration is less than 68 weeks. In certain embodiments, the longer treatment duration is greater than 68 weeks.

In certain embodiments, the sample and panel of clinical variables are obtained prior to the treatment regimen and/or at week 4, 12, 16, 20, 28, 36, 44, 52, 60, or 68 of treatment. In a specific embodiment, the sample and panel of clinical variables are obtained prior to the treatment regimen and again at week 68 of treatment.

In certain embodiments, the panel of clinical variables further comprises change in PASI.

Also provided for is a method of predicting a response to a treatment regimen with an anti-IL-23 antibody and treating for moderate to severe plaque psoriasis in a subject. The method can, for example, comprise:
a. obtaining a sample from the subject;
b. contacting the sample with a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A);
c. obtaining a panel of clinical variables from the subject comprising disease duration; body mass index (BMI), weight, age, sex, treatment history, Dermatology Life Quality Index (DLQI) score, and Psoriasis Area and Severity Index (PASI);
d. analyzing a range of threshold values of the panel of biomarkers and the panel of clinical variables to determine a predictive value for the subject, wherein a predictive value of less than about 0.1 indicates that the subject is less likely to be a super responder to the treatment regimen than a subject with a predictive value greater than about 0.1;
e. determining the treatment duration for the subject with the treatment regimen based on the predictive value, with a score of less than about 0.1 indicating that the subject will not be a super responder to treatment duration and a score of greater than about 0.1 indicating that the subject will be a super responder to the treatment regimen; and
f. treating the subject with the treatment regimen for a duration based on the score.

In a specific embodiment, a predictive value of 0 indicates that the subject is less likely to be a super responder to the treatment regimen than a subject with a predictive value of 1.

In certain embodiments, the subject has a score of greater than about 0.1, further comprising treating the subject with the anti-IL-23 antibody for a period of 68 weeks and ceasing treatment 68 weeks after initial treatment.

In a specific embodiment, the anti-IL-23 antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3.

In certain embodiments, the anti-IL-23 antibody comprises a light chain variable region amino acid sequence of SEQ ID NO: 8 and a heavy chain variable region amino acid sequence of SEQ ID NO: 7. In a specific embodiment, the anti-IL-23 antibody comprises a light chain amino acid sequence of SEQ ID NO: 10 and a heavy chain amino acid sequence of SEQ ID NO: 9. In a specific embodiment, the anti-IL-23 antibody is guselkumab.

In certain embodiments, the anti-IL-23 antibody is administered subcutaneously at a dose of 100 mg per administration. In certain embodiments, the antibody is administered in an initial dose, 4 weeks after the initial dose and every 8 weeks after the dose at 4 weeks. In certain embodiments, the antibody is administered every 8 or 16 weeks after a dose at 28 weeks.

Also provided is a kit for predicting a response to a treatment regimen for psoriasis in a subject. The kit can, for example, comprise:
a. an isolated set of probes capable of detecting a panel of biomarkers comprising at least one, such as 1, 2, 3, 4, 5, 6, 7, 8, 9, 10, 11, or more, biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A); and
b. instructions for use.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1G show the assessment of serum IL-17F (FIG. 1A), PI3 (FIG. 1D), CD163 (FIG. 1E), ITGB2 (FIG. 1F), ST2 (FIG. 1G), FGF-19 (FIG. 1B), and IL-10RA (FIG. 1C) levels in SRe who maintained drug-free disease control (PASI≤5) for >1 year after GUS withdrawal (reached W116) compared to SRes who lost disease control prior to W116 or to non-SRes (nSR). Level of IL-17F, PI3, CD163, integrin beta-2 (ITGB2), ST2, FGF19 and IL-10RA are plotted as mean with error bars representing 95% confidence intervals (n=288; 127 SRes and 161 non-SRes). Analyte levels from healthy control (HC) sera (n=55) are presented in the lower left corner for each protein. #: “Yes” vs “No” or “nSR” group p-value <0.05. “Yes” group: SRes who maintained drug-free disease control (PASI≤5) for >1 year after GUS withdrawal (reached W116). “No” group: SRes who lost disease control prior to W116

FIGS. 2A-2C show the 29 predictive variables identified with AUC of 0.944 with baseline serum biomarkers and broad clinical information (up to W68) as input variables. FIG. 2A shows a list of predictive variables identified in the order of relative importance. FIG. 2B shows an area under the curve plot. FIG. 2C shows a contingency table for the predictions of non-overlapping test set from 73 patients with PPV (Positive Predictive Value), NPV (Negative Predictive Value) and sensitivity/specificity are listed. “Yes” group: SRes who maintained drug-free disease control (PASI≤5) for >1 year after GUS withdrawal (reached W116). “No” group: SRes who lost disease control prior to W116.

FIG. 3 shows an example of two decision trees with 3 levels, that include range of the threshold value for each variable to split patient samples to each category (Leaf) in the independent test.

FIGS. 4A-C show 20 predictive variables identified with AUC of 0.833 with baseline serum biomarkers and early clinical information (up to W4) as input variables. FIG. 4A shows a list of predictive variables identified in the order of relative importance. FIG. 4B shows an area under the curve plot. FIG. 4C shows a contingency table for the predictions of non-overlapping test set from 73 patients with PPV (Positive Predictive Value), NPV (Negative Predictive Value) and sensitivity/specificity are listed. “Yes” group: SRes who maintained drug-free disease control (PASI≤5) for >1 year after GUS withdrawal (reached W116). “No” group: SRes who lost disease control prior to W116.

FIGS. 5A-5C show 18 predictive variables identified with AUC of 0.815 with baseline serum biomarkers and baseline clinical information as input variables. FIG. 5A shows a list of predictive variables identified in the order of relative importance. FIG. 5B shows an area under the curve plot. FIG. 5C shows a contingency table for the predictions of non-overlapping test set from 73 patients with PPV (Positive Predictive Value), NPV (Negative Predictive Value) and sensitivity/specificity are listed. “Yes” group: SRes who maintained drug-free disease control (PASI≤5) for >1 year after GUS withdrawal (reached W116). “No” group: SRes who lost disease control prior to W116.

FIGS. 6A-6C show 18 predictive variables identified with AUC of 0.610 with baseline serum biomarkers and baseline clinical information as input variables to predict Super Responders (SR) status at Week 28. FIG. 6A shows a list of predictive variables identified in the order of relative importance. FIG. 6B shows an area under the curve plot. FIG. 6C shows a contingency table for the predictions of non-overlapping test set from 73 patients with PPV (Positive Predictive Value), NPV (Negative Predictive Value) and sensitivity/specificity are listed. “SR” group: psoriasis patients who reached PASI score equal to 0 at Week 20 and Week 28 under Guselkumab treatment. “nSR” group: patients who did not reach PASI score of 0 at both Week 20 and Week 28 time point.

### DETAILED DESCRIPTION

The disclosed methods may be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended, and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed disclosure. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 10%, whichever is larger.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity.

As used herein, “biomarker” refers to a gene or protein whose level of expression or concentration in a sample is altered compared to that of a normal or healthy sample or is indicative of a condition. The biomarkers disclosed herein are genes and/or proteins whose expression level or concentration or timing of expression or concentration correlates with the capability of determining whether a subject is responsive to a biological therapy for psoriasis.

As used herein, “probe” refers to any molecule or agent that is capable of selectively binding to an intended target biomolecule. The target molecule can be a biomarker, for example, a nucleotide transcript or a protein encoded by or corresponding to a biomarker. Probes can be synthesized by one of skill in the art, or derived from appropriate biological preparations, in view of the present disclosure. Probes can be specifically designed to be labeled. Examples of molecules that can be utilized as probes include, but are not limited to, RNA, DNA, proteins, peptides, antibodies, aptamers, affibodies, and organic molecules.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

The term “administering” with respect to the methods of the disclosure, means a method for therapeutically or prophylactically preventing, treating or ameliorating a syndrome, disorder or disease (e.g., psoriasis) as described herein. Such methods include administering an effective amount of said therapeutic agent (e.g., an IL-23 therapeutic agent (e.g., guselkumab)) at different times during the course of a therapy or concurrently in a combination form. The methods of the disclosure are to be understood as embracing all known therapeutic treatment regimens.

The term “effective amount” means that amount of active compound or pharmaceutical agent that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., psoriasis).

### Biomarker Panel and Probes for Detecting the Biomarkers

The present disclosure relates generally to the prediction of responsiveness to a treatment regimen for psoriasis in a subject, and provides methods, reagents, and kits useful for this purpose. Provided herein are biomarkers that are predictive for responsiveness to a treatment regimen for psoriasis in a subject. In certain embodiments, the present disclosure provides a panel of biomarkers (e.g., genes that are expressed or proteins in a subject at a specific time point) that can be used to determine a treatment regimen or indicate the responsiveness to the treatment regimen for psoriasis.

Any methods available in the art for detecting expression of biomarkers are encompassed herein. The expression, presence, or amount of a biomarker of the disclosure can be detected on a nucleic acid level (e.g., as an RNA transcript) or a protein level. By “detecting or determining expression of a biomarker” is intended to include determining the quantity or presence of a protein or its RNA transcript for the biomarkers disclosed herein. Thus, “detecting expression” encompasses instances where a biomarker is determined not to be expressed, not to be detectably expressed, expressed at a low level, expressed at a normal level, or overexpressed.

In certain embodiments, provided herein are DNA-, RNA-, and protein-based diagnostic methods that either directly or indirectly detect the biomarkers described herein. The present disclosure also provides compositions, reagents, and kits for such diagnostic purposes. The diagnostic methods described herein may be qualitative or quantitative. Quantitative diagnostic methods may be used, for example, to compare a detected biomarker level to a cutoff or threshold level. Where applicable, qualitative or quantitative diagnostic methods can also include amplification of target, signal, or intermediary.

In certain embodiments, when utilizing a quantitative diagnostic method, an enrichment score is calculated. An enrichment score can be calculated utilizing gene set variation analysis (GSVA). GSVA is a non-parametric, unsupervised method for estimating variation of gene set enrichment through the samples of a gene expression dataset. The GSVA enrichment score is either the difference between the two sums or the maximum deviation from zero. Positive GSVA score indicates genes in the gene set of interest are positively enriched as compared to all other genes in the genome. Negative GSVA score means genes in the gene set of interest are negatively enriched as compared to genes not in the gene set.

In certain embodiments, biomarkers are detected at the nucleic acid (e.g., RNA) level. For example, the amount of biomarker RNA (e.g., mRNA) present in a sample is determined (e.g., to determine the level of biomarker expression). Biomarker nucleic acid (e.g., RNA, amplified cDNA, etc.) can be detected/quantified using a variety of nucleic acid techniques known to those of ordinary skill in the art, including but not limited to, nucleic acid hybridization and nucleic acid amplification.

In certain embodiments, a microarray is used to detect the biomarker. Microarrays can, for example, include DNA microarrays; protein microarrays; tissue microarrays; cell microarrays; chemical compound microarrays; and antibody microarrays. A DNA microarray, commonly referred to as a gene chip can be used to monitor expression levels of thousands of genes simultaneously. Microarrays can be used to identify disease genes by comparing expression in disease states versus normal states. Microarrays can also be used for diagnostic purposes, i.e., patterns of expression levels of genes can be studied in samples prior to the diagnosis of disease or after the diagnosis of disease (e.g., psoriasis), and these patterns can later be used to predict the treatment regimen for a disease in a subject at risk of or diagnosed with a disease or the responsiveness to a particular treatment regimen for a disease in a subject at risk of or diagnosed with a disease.

In certain embodiments, the expression products are proteins corresponding to the biomarkers of the panel. In certain embodiments detecting the levels of expression products comprises exposing the sample to antibodies for the proteins corresponding to the biomarkers of the panel. In certain embodiments, the antibodies are covalently linked to a solid surface. In certain embodiments, detecting the levels of expression products comprises exposing the sample to a mass analysis technique (e.g., mass spectrometry).

In certain embodiments, reagents are provided for the detection and/or quantification of biomarker proteins. The reagents can include, but are not limited to, primary antibodies that bind the protein biomarkers, secondary antibodies that bind the primary antibodies, affibodies that bind the protein biomarkers, aptamers (e.g., a SOMAmer) that bind the protein or nucleic acid biomarkers (e.g., RNA or DNA), and/or nucleic acids that bind the nucleic acid biomarkers (e.g., RNA or DNA). The detection reagents can be labeled (e.g., fluorescently) or unlabeled. Additionally, the detection reagents can be free in solution or immobilized.

In certain embodiments, when quantifying the level of a biomarker(s) present in a sample, the level can be determined on an absolute basis or a relative basis. When determined on a relative basis, comparisons can be made to controls, which can include, but are not limited to historical samples from the same patient (e.g., a series of samples over a certain time period), level(s) found in a subject or population of subjects without the disease or disorder (e.g., psoriasis), a threshold value, and an acceptable range.

Thus, provided herein are isolated sets of probes capable of detecting a panel of biomarkers, which are indicative of a responsiveness to a therapeutic regiment for a subject with psoriasis. In certain embodiments, provided is an isolated set of probes capable of detecting a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A).

In certain embodiments, the isolated set of probes is capable of detecting a panel of biomarkers comprising 1 or more, 2 or more, 3 or more, 4 or more, 5 or more, 6 or more, 7 or more, 8 or more, 9 or more, 10 or more, 11 or more biomarkers.

The probe can be any molecule or agent that specifically detects a biomarker. In certain embodiments, the probe is selected from the group consisting of an aptamer (such as a slow-off rate modified aptamer (SOMAmer)), an antibody, an affibody, a peptide, and a nucleic acid (such as an oligonucleotide hybridizing to the gene or mRNA of a biomarker). An aptamer is an oligonucleotide or a peptide that binds specifically to a target molecule. An aptamer is usually created by selection from a large random sequence pool. Examples of aptamers useful for the disclosure include oligonucleotides, such as DNA, RNA or nucleic acid analogues, or peptides, that bind to a biomarker of the disclosure. In one embodiment, the aptamers are single-stranded DNA-based protein affinity binding reagents, such as SOMAmers developed by SomaLogic, Inc. (Boulder, Colorado, USA). Under normal conditions (e.g., physiologic in serum), SOMAmers fold into specific shapes that bind target proteins with high affinity (sub-nM K d), but when SOMAmers are denatured, they can be detected and quantified by hybridizing to a standard DNA microarray. This dual nature of SOMAmers facilitates the detection of biomarkers that the SOMAmers specifically bind to.

### Machine Learning Modules

A computing device obtains the panel of biomarker values to generate a subject's response to a treatment regimen for psoriasis corresponding to the values of the biomarkers. The biomarker value may represent the amount of biomarker detected. Alternatively, the biomarker value may represent a binary status (yes/no) indicating whether the amount of is above a predetermined threshold value. The computing device may also obtain clinical variables of the subject, such as, for example, gender, age at week 0 of treatment, weight at week 0 of treatment, body mass index (BMI) at week 0 of treatment, disease duration, treatment history, Dermatology Life Quality Index (DLQI) score at week 0 of treatment, Psoriasis Area and Severity Index (PASI) at week 0, 4, 12, 16, 20, 28, 36, 44, 52, 60, or 68 of treatment, and change in PASI at week 4, 12, 16, 20, 28, 36, 44, 52, 60, or 68 of treatment. The computing device analyzes biomarker values and clinical values using a machine learning module to determine or predict whether the subject will respond to the treatment regimen. The machine learning module is trained using a set of reference data. The machine learning module compares the biomarker values and clinical values to a set of reference values to determine or predict whether the subject will respond to the treatment regimen. The set of reference data includes biomarker values and clinical values, along with the list of analytes in Appendix 1, for a reference group of subjects.

The machine learning module may be a supervised and/or unsupervised machine learning module. The machine learning module may be a machine learning classifier, for identifying dataset as correlating to one of two categories. The machine learning module may include support vector machine, random forest, logistic regression, gradient boosting module, or ensemble modules thereof. In one embodiment the machine learning module is an ensemble module comprising at least one of support vector machine, random forest, logistic regression, and/or gradient tree boosting module.

Those skilled in the art will understand that the exemplary computer-implemented embodiments described herein may be implemented in any number of manners, including as a separate software module, as a combination of hardware and software, etc. For example, the exemplary methods may be embodiment in one or more programs stored in a non-transitory storage medium and containing lines of code that, when compiled, may be executed by one or more processor cores or a separate processor. A system according to one embodiment comprises a plurality of processor cores and a set of instructions executing on the plurality of processor cores to perform the exemplary methods discussed above. The processor cores or separate processor may be incorporated in or may communicate with any suitable electronic device, for example, on board processing arrangements within the device or processing arrangements external to the device, e.g., a mobile computing device, a smart phone, a computing tablet, a computing device, etc., that may be in communications with at least a portion of the device.

### Therapeutic Applications

The present disclosure also provides a method for modulating or treating psoriasis, in a cell, tissue, organ, animal, or patient, as known in the art or as described herein, using at least one IL-23 antibody of the present disclosure, e.g., administering or contacting the cell, tissue, organ, animal, or patient with a therapeutic effective amount of IL-23 specific antibody.

In an embodiment, an anti-IL-23 antibody useful for the disclosure is a monoclonal antibody, preferably a human mAb, comprising heavy chain complementarity determining regions (CDRs) HCDR1, HCDR2, and HCDR3 of SEQ ID NOs: 1, 2, and 3, respectively; and light chain CDRs LCDR1, LCDR2, and LCDR3, of SEQ ID NOs: 4, 5, and 6, respectively.

The anti-IL-23 antibody can comprise at least one of a heavy or light chain variable region having a defined amino acid sequence. For example, in a preferred embodiment, the anti-IL-23 antibody comprises an anti-IL-23 antibody with a heavy chain variable region comprising an amino acid sequence at least 85%, preferably at least 90%, more preferably at least 95%, and most preferably 100% identical to SEQ ID NO: 7, and a light chain variable region comprising an amino acid sequence at least 85%, preferably at least 90%, more preferably at least 95%, and most preferably 100% identical to SEQ ID NO: 8. In an additional preferred embodiment, the anti-IL-23 antibody comprises at least one heavy chain, having the amino acid sequence of SEQ ID NO:9 and/or at least one light chain, having the amino acid sequence of SEQ ID NO: 10.

Preferably, the anti-IL-23 antibody is guselkumab (Tremfya®).

Another aspect of the method of the disclosure comprises administering a pharmaceutical composition comprising an isolated anti-IL-23 specific antibody as defined above, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state for use in the treatment of a patient.

Any method of the present disclosure can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluroquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2nd Edition, Appleton and Lange, Stamford, CT (2000); PDR Pharmacopoeia, Tarascon Pocket Pharmacopocia 2000, Deluxe Edition, Tarascon Publishing, Loma Linda, CA (2000); Nursing 2001 Handbook of Drugs, 21st edition, Springhouse Corp., Springhouse, PA, 2001; Health Professional's Drug Guide 2001, ed., Shannon, Wilson, Stang, Prentice-Hall, Inc, Upper Saddle River, NJ, each of which references are entirely incorporated herein by reference.

Typically, treatment of psoriasis is affected by administering an effective amount or dosage of an anti-IL-23 antibody composition that total, on average, a range from at least about 0.01 to 500 milligrams of an anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present disclosure 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Alternatively or additionally, treatment of humans of animals can be provided as a periodic dosage of at least one antibody of the present disclosure per week on at least one of week 4, 12, 20, 28, 36, 44, 52, 60, 68, 80, 92, 104, or 116 or any combination thereof.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

### Kits

Also provided are kits for predicting a response to a treatment regimen for an psoriasis in a subject. The kits can, for example, comprise (a) an isolated set of probes capable of detecting a panel of biomarkers comprising at least one, such as 1, 2, 3, 4, 5, 6, 7, 8, 9, 10, 11, or more, biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A); and (b) instructions for use.

Compositions for use in the methods disclosed herein include, but are not limited to, probes, antibodies, affibodies, nucleic acids, and/or aptamers. Preferred compositions can detect the level of expression (e.g., mRNA or protein level) of a panel of biomarkers from a biological sample.

Any of the compositions can be provided in the form of a kit or a reagent mixture. By way of an example, labeled probes can be provided in a kit for the detection of a panel of biomarkers. Kits can include all components necessary or sufficient for assays, which can include, but is not limited to, detection reagents (e.g., probes), buffers, control reagents (e.g., positive and negative controls), amplification reagents, solid supports, labels, instruction manuals, etc. In certain embodiments, the kit comprises a set of probes for the panel of biomarkers and a solid support to immobilize the set of probes. In certain embodiments, the kit comprises a set of probes for the panel of biomarkers, a solid support, and reagents for processing the sample to be tested (e.g., reagents to isolate the protein or nucleic acids from the sample).

### Embodiments

The disclosure provides the following non-limiting embodiments.

Embodiment 1 is a method of predicting a response to a treatment regimen for psoriasis in a subject in need thereof, the method comprising:
a. obtaining a sample from the subject;
b. contacting the sample with a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A);
c. obtaining a panel of clinical variables from the subject comprising disease duration, body mass index (BMI), weight, age, sex, treatment history, Dermatology Life Quality Index (DLQI) score, and Psoriasis Area and Severity Index (PASI);
d. analyzing a range of threshold values of the panel of biomarkers and the panel of clinical variables to determine a predictive value for the subject, wherein a predictive value of less than about 0.1 indicates that the subject is less likely to respond to the treatment regimen than a subject with a predictive value greater than about 0.1;
e. determining the treatment duration for the subject with the treatment regimen based on the predictive value, with a score of greater than about 0.1 indicating treating the subject for a shorter duration and a score of less than about 0.1 indicating treating the subject for a longer duration; and
f. treating the subject with the treatment regimen for a duration based on the score.

Embodiment 2 is the method of embodiment 1, wherein the contacting step comprises contacting the samples with an isolated set of probes corresponding to the panel of biomarkers.

Embodiment 3 is the method of embodiment 2, wherein the sample is a blood sample.

Embodiment 4 is the method of embodiment 1, wherein the method further comprises administering a therapeutic agent to the subject to treat or prevent the psoriasis.

Embodiment 5 is the method of embodiment 1, wherein the therapeutic agent is an anti-IL-23 antibody.

Embodiment 6 is the method of embodiment 5, wherein the anti-IL-23 antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3.

Embodiment 7 is the method of embodiment 5, wherein the anti-IL-23 antibody comprises a light chain variable region amino acid sequence of SEQ ID NO: 8 and a heavy chain variable region amino acid sequence of SEQ ID NO: 7.

Embodiment 8 is the method of embodiment 5, wherein the anti-IL-23 antibody comprises a light chain amino acid sequence of SEQ ID NO: 10 and a heavy chain amino acid sequence of SEQ ID NO: 9.

Embodiment 9 is the method of embodiment 5, wherein the anti-IL-23 antibody is guselkumab.

Embodiment 10 is the method of embodiments 5-9, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

Embodiment 11 is the method of embodiment 1, wherein the analyzing step is performed using a machine learning module.

Embodiment 12 is the method of embodiment 11, wherein the machine learning model comprises at least one of a support vector machine module, a random forest module, a logistic regression module, and a gradient tree boosting module.

Embodiment 13 is the method of embodiment 1, wherein the shorter treatment duration is less than 68 weeks.

Embodiment 14 is the method of embodiment 1, wherein the longer treatment duration is greater than 68 weeks.

Embodiment 15 is the method of any of embodiments 1 to 10, wherein the sample and panel of clinical variables are obtained prior to the treatment regimen and/or at week 4, 12, 16, 20, 28, 36, 44, 52, 60, or 68 of treatment.

Embodiment 16 is the method of embodiment 1, wherein the panel of clinical variables further comprises change in PASI.

Embodiment 17 is a method of predicting a response to a treatment regimen with an anti-IL-23 antibody and treating for moderate to severe plaque psoriasis in a subject in need thereof, the method comprising:
a. obtaining a sample from the subject;
b. contacting the sample with a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A);
c. obtaining a panel of clinical variables from the subject comprising disease duration, body mass index (BMI), weight, age, sex, treatment history, Dermatology Life Quality Index (DLQI) score, and Psoriasis Area and Severity Index (PASI);
d. analyzing a range of threshold values of the panel of biomarkers and the panel of clinical variables to determine a predictive value for the subject, wherein a predictive value of less than about 0.1 indicates that the subject is less likely to be a super responder to the treatment regimen than a subject with a predictive value greater than about 0.1;
e. determining the treatment duration for the subject with the treatment regimen based on the predictive value, with a score of less about 0.1 indicating that the subject will not be a super responder to treatment duration and a score of greater than about 0.1 indicating that the subject will be a super responder to the treatment regimen; and
f. treating the subject with the treatment regimen for a duration based on the score.

Embodiment 18 is the method of embodiment 17, wherein the has subject a score of greater than about 0.1, further comprising treating the subject with the anti-IL-23 antibody for a period of 68 weeks and ceasing treatment 68 weeks after initial treatment.

Embodiment 19 is the method of embodiment 17 or 18, wherein the anti-IL-23 antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3.

Embodiment 20 is the method of embodiment 17 or 18, wherein the anti-IL-23 antibody comprises a light chain variable region amino acid sequence of SEQ ID NO: 8 and a heavy chain variable region amino acid sequence of SEQ ID NO: 7.

Embodiment 21 is the method of embodiment 17 or 18, wherein the anti-IL-23 antibody comprises a light chain amino acid sequence of SEQ ID NO: 10 and a heavy chain amino acid sequence of SEQ ID NO: 9.

Embodiment 22 is the method of embodiment 17 or 18, wherein the anti-IL-23 antibody is guselkumab.

Embodiment 23 is the method of embodiment 17-22, wherein the anti-IL-23 antibody is administered subcutaneously at a dose of 100 mg per administration.

Embodiment 24 is the method of embodiment 23, wherein the antibody is administered in an initial dose, 4 weeks after the initial dose and every 8 weeks after the dose at 4 weeks.

Embodiment 25 is the method of embodiment 24, wherein the antibody is administered every 8 or 16 weeks after a dose at 28 weeks.

Embodiment 26 is the method of any of embodiments 1 to 25, wherein a predictive value of 0 indicates that the subject is less likely to respond to the treatment regimen than a subject with a predictive value of 1.

Embodiment 27 is a kit for predicting a response to a treatment regimen for psoriasis in a subject in need thereof, the kit comprising:
a. an isolated set of probes capable of detecting a panel of biomarkers comprising at least one, such as 1, 2, 3, 4, 5, 6, 7, 8, 9, 10, 11, or more, biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A); and
b. instructions for use.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the disclosure.

### Example 1

GUIDE is an ongoing phase III study that examines clinical and immunological impact of new treatment strategies with GUS in patients with moderate-to-severe plaque-type psoriasis (PSO). In GUIDE, subjects who achieved PASI-0 at both week (W) 20 and W28 were defined as super responders (SRe); all other subjects were labeled as non-SRe at W28. SRes with PASI<3 at W68 were withdrawn from treatment in part 3 of the study (W68-220). Subjects were monitored to see if they were able to maintain drug-free disease control (PASI≤5) following GUS withdrawal.

To identify potential features for predicting SRe who can maintain disease control (PASI≤5) for >1 year after GUS withdrawal, broad serum proteomic analysis was performed on 288 subjects (127 SRes, 161 non-SRes). We identified baseline serum biomarkers that were significantly higher or lower in SRes who maintained drug-free disease control for >1 year after GUS withdrawal (reached W116), compared to SRes who lost disease control prior to W116 or to non-SRes. Additional analysis utilizing a machine-learning decision tree algorithm and using serum and clinical features identified combination of features that are predictive for a patient becoming SRe who can maintain drug-free disease control for >1 year after GUS withdrawal.

SRes who maintained drug-free disease control (PASI≤5) for >1 year after GUS withdrawal (reached W116) were characterized by significantly lower levels of elafin/peptidase inhibitor 3 (PI3), scavenger receptor cysteine-rich type 1 protein M130 (CD163), integrin beta-2 (ITGB2), suppression of tumorigenicity 2 protein (ST2), IL-interleukin 17F (IL-17F), and significantly higher levels of fibroblast growth factor 19 (FGF19) and interleukin-10 receptor subunit alpha (IL-10RA) at baseline, compared to SRes who lost disease control prior to W116 or to non-SRes

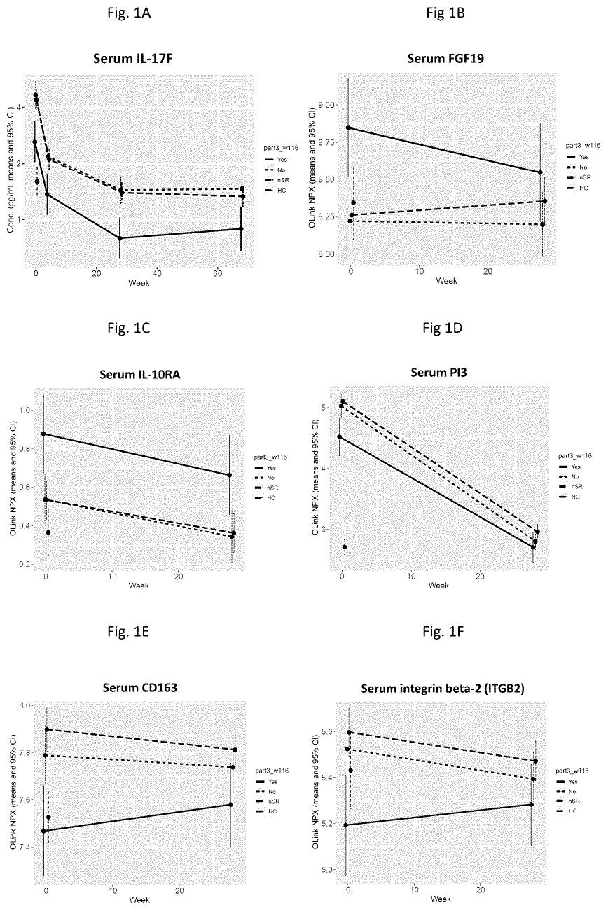

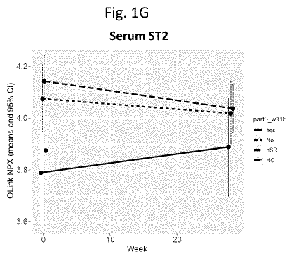

To identify potential predictive serum biomarkers for maintaining disease control (PASI≤5) for >1 year after GUS withdrawal, broad serum proteomic analysis was performed on 288 subjects (127 SRes and 161 non-SRes). Serum level of interleukin 17A (IL-17A), IL-17F, IL-22, beta-defensin-2 (BD-2) and IL-19, which are proteins downstream of IL-23 pathway and have been demonstrated to be upregulated in PSO subjects, were analyzed at the single analyte level. An additional 276 analytes were evaluated using Olink Target 96 platform (Cardiovascular II, Cardiovascular III and Inflammation panels). To focus our objective on identifying potential predictive biomarkers, we evaluated baseline serum level in SRes who maintained drug-free disease control for >1 year (reached W116) compared to SRes who lost disease control prior to W116 or to non-SRes. Our analysis identified that level of IL-17F, elafin/peptidase inhibitor 3 (PI3), scavenger receptor cysteine-rich type 1 protein M130 (CD163), integrin beta-2 (ITGB2), suppression of tumorigenicity 2 protein (ST2) was significantly lower, while levels of fibroblast growth factor 19 (FGF19) and interleukin-10 receptor subunit alpha (IL-10RA) were significantly higher in SRes who maintained drug-free disease control for >1 year compared to SRes who lost disease control prior to W116 or to non-SRes (FIG. 1). List of all analytes evaluated, and their corresponding measurement platforms are summarized in Appendix 1.

### Example 2

Additional analysis utilizing machine-learning decision tree algorithm was performed to identify combination of baseline biomarkers and clinical information that are predictive for a patient becoming SRe and being able to maintain drug-free disease control for >1 year after GUS withdrawal.

A publicly available R package (XGBoost: https://cran.r-project.org/web/packages/xgboost/index.html) was used to predict SRes who can maintain drug-free disease control for >1 year after GUS withdrawal. Overall, this method used an efficient implementation of the gradient boosting learning framework from Chen & Guestrin (2016) <doi: 10.1145/2939672.2939785> to identify an ensemble/group of decision trees on the values of clinical and serum biomarkers to obtain an optimal prediction of patients' part 3 status, i.e. whether they become super responders who can maintain drug-free responses with (PASI score≤5) for >1 year after GUS withdrawal.

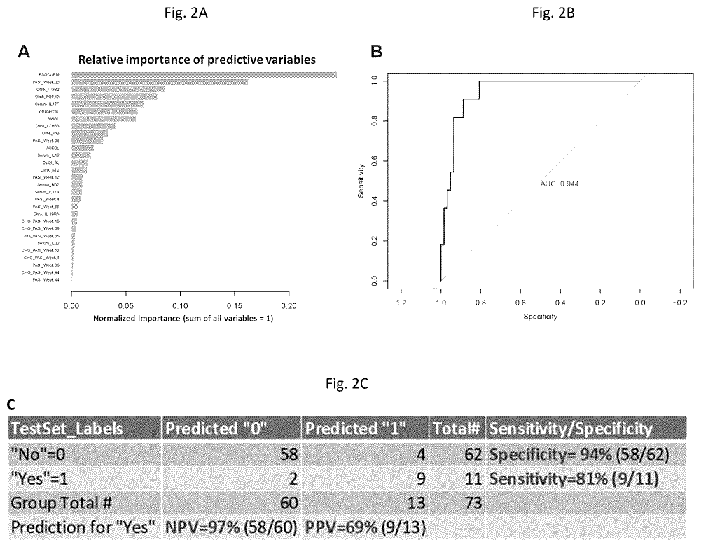

Baseline serum biomarker data included analytes measured at the single level (IL-17A, IL-17F, IL-22, BD-2 and IL-19) and analytes from Olink analysis that were identified to be significantly higher/lower in SRe who maintained drug-free disease control (PASI≤5) for >1 year after GUS withdrawal (reached W116) compared to SRes who lost disease control prior to W116 or to non-SRes: PI3, CD163, integrin beta-2 (ITGB2), ST2, FGF19 and IL-10RA. 28 clinical variables (up to W68) were included in the analysis as summarized in Table 1. Biomarker data from 75% of the samples (220 patients) were used to train the predictive model and the rest of non-overlapping 25% samples (73 patients) were used as test set to evaluate model prediction performance. This analysis identified 29 variables (Table 2) that are predictive for a patient becoming SRe who can maintain drug-free disease control (PASI≤5) for >1 year after GUS withdrawal with AUC of 0.944 (FIG. 2). This model successfully identified 9 out 11 SRes who maintained drug-free disease control (PASI≤5) for >1 year after GUS withdrawal (reached W116) among the non-SRes and SRes who lost disease control prior to W116. Threshold values for each identified variables is also summarized in Table 2.

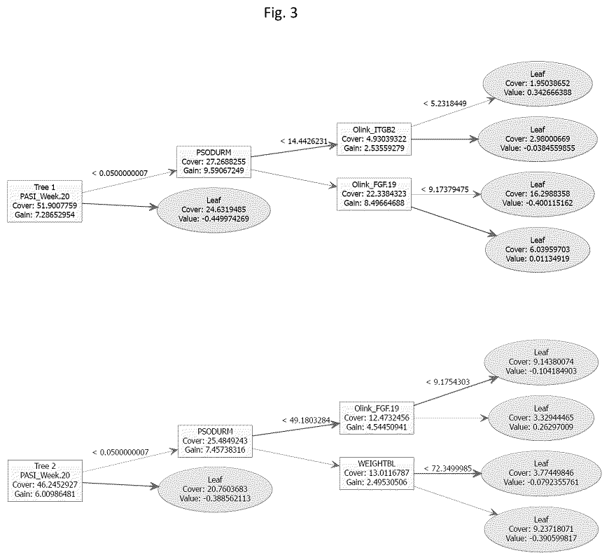

Example of decision tree with 3 levels, that include the threshold value for each variable to split patient samples to each category (Leaf) are shown in FIG. 3. Final prediction was determined by averaged predictive scores from each leaf node.

TABLE 1
List of 28 clinical variables included in
machine learning decision tree algorithm analysis.
Clinical Variables

Disease duration
Gender
Age at BL (baseline)
Treatment history (prior biologics or
not)
DLQI at BL
Weight at BL
BMI at BL
PASI at BL
PASI at W 4
PASI at W 12
PASI at W 16
PASI at W 20
PASI at W 28
PASI at W 36
PASI at W 44
PASI at W 52
PASI at W 60
PASI at W 68
Change in PASI at W 4
Change in PASI at W 12
Change in PASI at W 16
Change in PASI at W 20
Change in PASI at W 28
Change in PASI at W 36
Change in PASI at W 44
Change in PASI at W 52
Change in PASI at W 60
Change in PASI at W 68

TABLE 2
List of identified variables in the order of relative importance
and the threshold values for model using 11 baseline serum analyte
levels and 28 clinical variables (up to W 68 clinical response)

| Variables | Threshold Values | | Category* |
|---|---|---|---|
| PSO disease duration | 14 to 50 | months | Below |
| PASI at W 20 | 0.05 | | Below |
| Baseline ITGB2 | 5.2 | NPX | Below |
| Baseline FGF-19 | 8.4 | NPX | Above |
| Baseline IL-17F | 2 | pg/ml | Below |
| Baseline weight | 72 | kg | Below |
| Baseline BMI | 24 | | Below |
| Baseline CD163 | 7.26 | NPX | Below |
| Baseline PI3 | 4.19 | NPX | Below |
| PASI at W 28 | 0.1 | | Below |
| Baseline age | 30 to 39 | years old | Below |
| Baseline IL-19 | 5.4 | pg/ml | Below |
| Baseline DLQI score | 16.5 | | Below |
| Baseline ST2 | 3.81 | NPX | Below |
| PASI at W 12 | 0.75 | | Below |
| Baseline BD-2 | 10.3 | pg/ml | Below |
| Baseline IL-17A | 0.8 | pg/ml | Below |
| PASI at W 4 | 4.2 | | Below |
| PASI at W 68 | 5.5 | | Below |
| Baseline IL-10RA | 0.17 | NPX | Above |
| Change in PASI at W 16 | −16.85 | | Above |
| Change in PASI at W 68 | −17 | | Above |
| Change in PASI at W 36 | −31.1 | | Above |
| Baseline IL-22 | 12.1 | pg/ml | Below |

| | | |
|---|---|---|
| Change in PASI at W 12 | −16.35 | Above |
| Change in PASI at W 4 | −10.8 | Above |
| PASI at W 36 | 0.05 | Below |
| Change in PASI at W 44 | −13.95 | Above |
| PASI at W 44 | 1.05 | Below |

*For Category column:
Above: >threshold value/range of threshold values indicate higher likelihood of becoming SRes who can maintain drug-free disease control (PASI ≤ 5) for >1 year after GUS withdrawal
Below: <threshold value/range of threshold values indicate higher likelihood of becoming SRes who can maintain drug-free disease control (PASI ≤ 5) for >1 year after GUS withdrawal

### Example 3

Analysis using machine-learning decision tree algorithm using 11 baseline biomarker level and 10 clinical variables (up to W4 clinical response) identify 20 variables that are predictive for a patient becoming SRe and being able to maintain drug-free disease control (PASI≤5) for >1 year after GUS withdrawal with AUC of 0.833

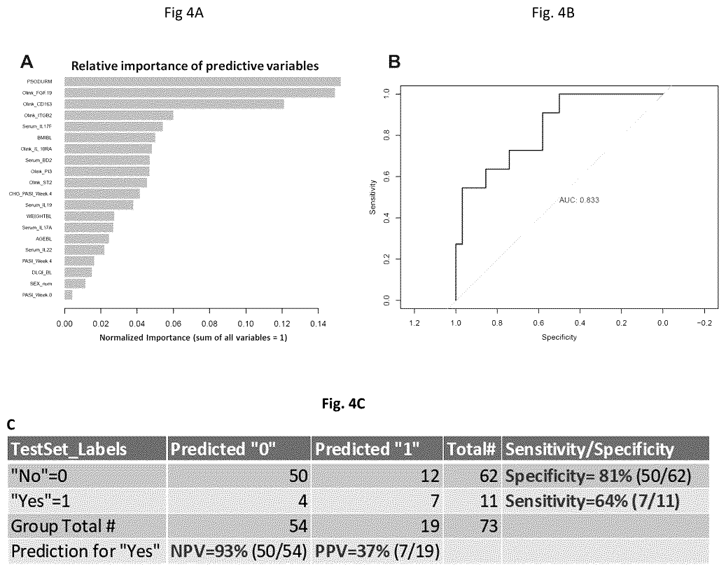

Analysis using machine-learning decision tree algorithm was performed again as described in Example 2 using same baseline serum biomarker data (IL-17A, IL-17F, IL-22, BD-2, IL-19, PI3, CD163, integrin beta-2 (ITGB2), ST2, FGF19 and IL-10RA), but with only 10 clinical variables (up to W4) as summarized in Table 3. Biomarker data from 75% of the samples (220 patients) were used to train the predictive model and the rest of non-overlapping 25% samples (73 patients) were used as test set to evaluate model prediction performance. This analysis identified 20 variables (Table 4) that are predictive for a patient becoming SRe who can maintain drug-free disease control (PASI≤5) for >1 year after GUS withdrawal with AUC of 0.833 (FIG. 4). This model successfully identified 7 out 11 SRes who maintained drug-free disease control (PASI≤5) for >1 year after GUS withdrawal (reached W116) among the non-SRes and SRes who lost disease control prior to W116. Threshold values for each identified variables is also summarized in Table 4.

TABLE 3
List of 10 clinical variables included in
machine learning decision tree algorithm analysis.
Clinical Variables

| | |
|---|---|
| Disease duration | Weight at BL |
| Gender | BMI at BL |
| Age at BL (baseline) | PASI at BL |
| Treatment history (prior biologics or | PASI at W 4 |
| not) | |
| DLQI at BL | Change in PASI at W 4 |

TABLE 4
List of identified variables in the order of relative importance
and the threshold values for model using 11 baseline biomarker
level and 10 clinical variables (up to W 4 clinical response)

| Variables | Threshold Values | | Category* |
|---|---|---|---|
| PSO disease duration | 14 to 50 | months | Below |
| Baseline FGF19 | 8.4 | NPX | Above |
| Baseline CD163 | 7.26 | NPX | Below |
| Baseline ITGB2 | 5.2 | NPX | Below |
| Baseline IL-17F | 2 | pg/ml | Below |
| Baseline BMI | 24 | | Below |
| Baseline IL-10RA | 0.17 | NPX | Above |
| Baseline BD-2 | 10.3 | pg/ml | Below |
| Baseline PI3 | 4.19 | NPX | Below |
| Baseline ST2 | 3.81 | NPX | Below |
| Change in PASI at W 4 | −10.8 | | Above |
| Baseline IL-19 | 5.4 | pg/ml | Below |
| Baseline weight | 72 | kg | Below |
| Baseline IL-17A | 0.8 | pg/ml | Below |
| Baseline age | 30 to 39 | years old | Below |
| Baseline IL-22 | 12.1 | pg/ml | Below |

| | | |
|---|---|---|
| PASI at W 4 | 4.2 | Below |
| Baseline DLQI score | 16.5 | Below |
| Gender | Not Applicable | Not Applicable |
| PASI at W 0 | 19.4 | Below |

*For Category column:
Above: >threshold value/range of threshold values indicate higher likelihood of becoming SRes who can maintain drug-free disease control (PASI ≤ 5) for >1 year after GUS withdrawal
Below: <threshold value/range of threshold values indicate higher likelihood of becoming SRes who can maintain drug-free disease control (PASI ≤ 5) for >1 year after GUS withdrawal

### Example 4

Analysis using machine-learning decision tree algorithm using 11 baseline biomarker level and 8 clinical variables (baseline only) identify 18 variables that are predictive for a patient becoming SRe and being able to maintain drug-free disease control (PASI≤5) for >1 year after GUS withdrawal with AUC of 0.815

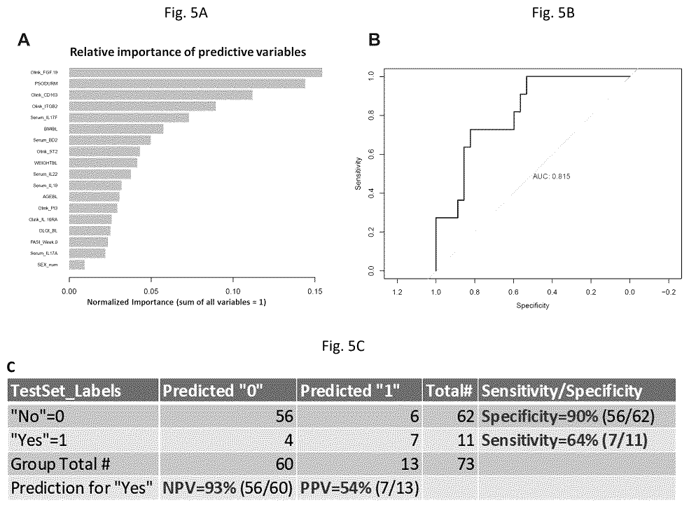

Analysis using machine-learning decision tree algorithm was performed again as described in Example 2 and Example 3 using same baseline serum biomarker (IL-17A, IL-17F, IL-22, BD-2, IL-19, PI3, CD163, integrin beta-2 (ITGB2), ST2, FGF19 and IL-10RA), but with only 8 clinical variables (baseline) as summarized in Table 5. Biomarker data from 75% of the samples (220 patients) were used to train the predictive model and the rest of non-overlapping 25% samples (73 patients) were used as test set to evaluate model prediction performance. This analysis identified 18 variables (Table 6) that are predictive for a patient becoming SRe who can maintain drug-free disease control (PASI≤5) for >1 year after GUS withdrawal with AUC of 0.815 (FIG. 5). This model successfully identified 7 out 11 SRes who maintained drug-free disease control (PASI≤5) for >1 year after GUS withdrawal (reached W116) among the non-SRes and SRes who lost disease control prior to W116. Threshold values for each identified variables is also summarized in Table 6.

TABLE 5
List of 10 clinical variables included in
machine learning decision tree algorithm analysis.
Clinical Variables

| | |
|---|---|
| Disease duration | DLQI at BL |
| Gender | Weight at BL |
| Age at BL (baseline) | BMI at BL |
| Treatment history (prior biologics or | PASI at BL |
| not) | |

TABLE 6
List of identified variables in the order of relative importance
and the threshold values for model using 11 baseline biomarker
level and 8 clinical variables (baseline only)

| Variables | Threshold Values | | Category* |
|---|---|---|---|
| Baseline FGF19 | 8.4 | NPX | Above |
| PSO disease duration | 14 to 50 | months | Below |
| Baseline CD163 | 7.26 | NPX | Below |
| Baseline ITGB2 | 5.2 | NPX | Below |
| Baseline IL-17F | 2 | pg/mL | Below |
| Baseline BMI | 24 | | Below |
| Baseline BD-2 | 10.3 | pg/mL | Below |
| Baseline ST2 | 3.81 | NPX | Below |
| Baseline weight | 72 | kg | Below |
| Baseline IL-22 | 12.1 | pg/mL | Below |
| Baseline IL-19 | 5.4 | pg/mL | Below |
| Baseline age | 30 to 39 | years old | Below |
| Baseline PI3 | 4.19 | NPX | Below |
| Baseline IL-10RA | 0.17 | NPX | Above |
| Baseline DLQI score | 16.5 | | Below |
| PASI at W 0 | 19.4 | | Below |
| Baseline IL-17A | 0.8 | pg/mL | Below |

| | | |
|---|---|---|
| Gender | Not Applicable | Not Applicable |

*For Category column:
Above: >threshold value/range of threshold values indicate higher likelihood of becoming SRes who can maintain drug-free disease control (PASI ≤ 5) for >1 year after GUS withdrawal
Below: <threshold value/range of threshold values indicate higher likelihood of becoming SRes who can maintain drug-free disease control (PASI ≤ 5) for >1 year after GUS withdrawal

### Example 5

Analysis using machine-learning decision tree algorithm on 11 baseline serum analyte levels and 8 clinical variables (baseline only) identified 18 features that in combination are predictive for a patient becoming SRe with AUC of 0.61

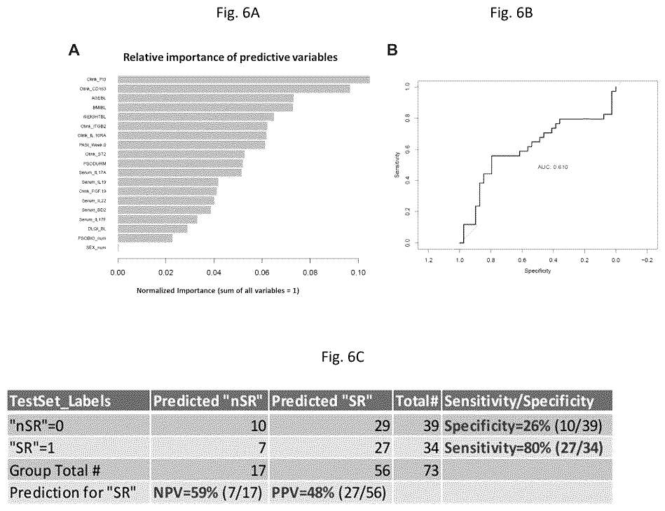

Analysis using machine-learning decision tree algorithm was performed again as described in Example 2, 3 and 4, using same baseline serum biomarker (IL-17A, IL-17F, IL-22, BD-2, IL-19, PI3, CD163, integrin beta-2 (ITGB2), ST2, FGF19 and IL-10RA), and with only 8 clinical variables (baseline) as summarized in Table 7. Biomarker data from 75% of the samples (220 patients) were used to train the predictive model and the rest of non-overlapping 25% samples (73 patients) were used as test set to evaluate model prediction performance. This analysis identified 18 variables (Table 8) that are predictive for a patient becoming SRe with AUC of 0.61 (FIG. 6). This model successfully identified 27 out 34 SRes among the non-SRes (corresponding to 80% sensitivity). Threshold values for each identified variables is also summarized in Table 8.

TABLE 7
List of 8 clinical variables included in
machine learning decision tree algorithm analysis.
Clinical Variables

| | |
|---|---|
| Disease duration | DLQI at BL |
| Gender | Weight at BL |
| Age at BL (baseline) | BMI at BL |
| Treatment history (prior biologics or | PASI at BL |
| not) | |

TABLE 8
List of identified variables in the order of relative
importance and the threshold values for model using 11
baseline biomarker level and 8 baseline clinical variables
to predict Super-Responder (SRe) status at week 28

| Variables | Threshold Values | | Category* |
|---|---|---|---|
| Baseline PI3 | 6.3 | NPX | Below |
| Baseline CD163 | 7.5 | NPX | Below |
| Baseline age | 45 | | Below |
| Baseline BMI | 24.3 | | Below |
| Baseline weight | 75 | kg | Below |
| Baseline ITGB2 | 7.4 | NPX | Below |
| Baseline IL-10RA | 0.67 | NPX | Above |
| PASI at W 0 | 15.4 | | Below |
| Baseline ST2 | 3.3 | NPX | Below |
| PSO disease duration | 22 | month | Below |
| Baseline IL-17A | 0.66 | pg/ml | Below |
| Baseline IL-19 | 45 | pg/ml | Below |
| Baseline FGF19 | 8.4 | NPX | Above |
| Baseline IL-22 | 6.8 | pg/ml | Below |
| Baseline BD-2 | 23.7 | pg/ml | Below |
| Baseline IL-17F | 3.2 | pg/ml | Below |

| | | |
|---|---|---|
| Baseline DLQI score | 18.5 | Below |
| Number of prior biologic | 1 | Below |
| treatment | | |

*For Category column:
Above: >threshold value/range of threshold values indicate higher likelihood of becoming SRes
Below: <threshold value/range of threshold values indicate higher likelihood of becoming SRes

APPENDIX 1
List of all analytes evaluated and corresponding measurement platform

| Analytes | Platform |
|---|---|
| Interleukin 17A (IL-17A) | S-plex assay, MSD |
| Interleukin 17F (IL-17F) | SMC assay, Millipore |
| Interleukin 22 (IL-22) | SMC assay, Millipore |
| Interleukin 19 (IL-19) | ELISA assay, R&D |
| Beta-defensin-2 (BD-2) | U-plex assay, MSD |
| 2,4-dienoyl-CoA reductase, mitochondrial (DECR1) | Target 96 Cardiovascular II |
| | panel, Olink |
| A disintegrin and metalloproteinase with | Target 96 Cardiovascular II |
| thrombospondin motifs 13 (ADAM-TS13) | panel, Olink |
| ADM (ADM) | Target 96 Cardiovascular II |
| | panel, Olink |
| Agouti-related protein (AGRP) | Target 96 Cardiovascular II |
| | panel, Olink |
| Alpha-L-iduronidase (IDUA) | Target 96 Cardiovascular II |
| | panel, Olink |
| Angiopoietin-1 (ANG-1) | Target 96 Cardiovascular II |
| | panel, Olink |
| Angiopoietin-1 receptor (TIE2) | Target 96 Cardiovascular II |
| | panel, Olink |
| Angiotensin-converting enzyme 2 (ACE2) | Target 96 Cardiovascular II |
| | panel, Olink |
| Bone morphogenetic protein 6 (BMP-6) | Target 96 Cardiovascular II |
| | panel, Olink |
| Brother of CDO (Protein BOC) | Target 96 Cardiovascular II |
| | panel, Olink |
| Carbonic anhydrase 5A, mitochondrial (CA5A) | Target 96 Cardiovascular II |
| | panel, Olink |
| Carcinoembryonic antigenrelated cell adhesion | Target 96 Cardiovascular II |
| molecule 8 (CEACAM8) | panel, Olink |
| Cathepsin L1 (CTSL1) | Target 96 Cardiovascular II |
| | panel, Olink |
| C-C motif chemokine 3 (CCL3) | Target 96 Cardiovascular II |
| | panel, Olink |
| C-C motif chemokine 17 (CCL17) | Target 96 Cardiovascular II |
| | panel, Olink |
| CD40 ligand (CD40-L) | Target 96 Cardiovascular II |
| | panel, Olink |
| Chymotrypsin C (CTRC) | Target 96 Cardiovascular II |
| | panel, Olink |
| C-X-C motif chemokine 1 (CXCL1) | Target 96 Cardiovascular II |
| | panel, Olink |
| Decorin (DCN) | Target 96 Cardiovascular II |
| | panel, Olink |
| Dickkopf-related protein 1 (Dkk-1) | Target 96 Cardiovascular II |
| | panel, Olink |
| Fatty acid-binding protein, intestinal (FABP2) | Target 96 Cardiovascular II |
| | panel, Olink |
| Fibroblast growth factor 21 (FGF-21) | Target 96 Cardiovascular II |
| | panel, Olink |
| Fibroblast growth factor 23 (FGF-23) | Target 96 Cardiovascular II |
| | panel, Olink |
| Follistatin (FS) | Target 96 Cardiovascular II |
| | panel, Olink |
| Galectin-9 (Gal-9) | Target 96 Cardiovascular II |
| | panel, Olink |
| Gastric intrinsic factor (GIF) | Target 96 Cardiovascular II |
| | panel, Olink |
| Gastrotropin (GT) | Target 96 Cardiovascular II |
| | panel, Olink |
| Growth hormone (GH) | Target 96 Cardiovascular II |
| | panel, Olink |
| Growth/differentiation factor 2 (GDF-2) | Target 96 Cardiovascular II |
| | panel, Olink |
| Heat shock 27 kDa protein (HSP 27) | Target 96 Cardiovascular II |
| | panel, Olink |
| Heme oxygenase 1 (HO-1) | Target 96 Cardiovascular II |
| | panel, Olink |
| Hydroxyacid oxidase 1 (HAOX1) | Target 96 Cardiovascular II |
| | panel, Olink |
| Interleukin-1 receptor antagonist protein (IL-1ra) | Target 96 Cardiovascular II |
| | panel, Olink |
| Interleukin-1 receptor-like 2 (IL1RL2) | Target 96 Cardiovascular II |
| | panel, Olink |
| Interleukin-4 receptor subunit alpha (IL-4RA) | Target 96 Cardiovascular II |
| | panel, Olink |
| Interleukin-6 (IL6) | Target 96 Cardiovascular II |
| | panel, Olink |
| Interleukin-17D (IL-17D) | Target 96 Cardiovascular II |
| | panel, Olink |
| Interleukin-18 (IL-18) | Target 96 Cardiovascular II |
| | panel, Olink |
| Interleukin-27 (IL-27) | Target 96 Cardiovascular II |
| | panel, Olink |
| Kidney Injury Molecule (KIM1) | Target 96 Cardiovascular II |
| | panel, Olink |
| Lactoylglutathione lyase (GLO1) | Target 96 Cardiovascular II |
| | panel, Olink |
| Lectin-like oxidized LDL receptor 1 (LOX-1) | Target 96 Cardiovascular II |
| | panel, Olink |
| Leptin (LEP) | Target 96 Cardiovascular II |
| | panel, Olink |
| Lipoprotein lipase (LPL) | Target 96 Cardiovascular II |
| | panel, Olink |
| Low affinity immunoglobulin gamma Fc region | Target 96 Cardiovascular II |
| receptor II-b (IgG Fc receptor II-b) | panel, Olink |
| Lymphotactin (XCL1) | Target 96 Cardiovascular II |
| | panel, Olink |
| Macrophage receptor MARCO (MARCO) | Target 96 Cardiovascular II |
| | panel, Olink |
| Matrix metalloproteinase-7 (MMP-7) | Target 96 Cardiovascular II |
| | panel, Olink |
| Matrix metalloproteinase-12 (MMP-12) | Target 96 Cardiovascular II |
| | panel, Olink |
| Melusin (ITGB1BP2) | Target 96 Cardiovascular II |
| | panel, Olink |
| Natriuretic peptides B (BNP) | Target 96 Cardiovascular II |
| | panel, Olink |
| NF-kappa-B essential modulator (NEMO) | Target 96 Cardiovascular II |
| | panel, Olink |
| Osteoclast-associated immunoglobulin-like receptor | Target 96 Cardiovascular II |
| (hOSCAR) | panel, Olink |
| Pappalysin-1 (PAPPA) | Target 96 Cardiovascular II |
| | panel, Olink |
| Pentraxin-related protein PTX3 (PTX3) | Target 96 Cardiovascular II |
| | panel, Olink |
| Placenta growth factor (PGF) | Target 96 Cardiovascular II |
| | panel, Olink |
| Platelet-derived growth factor subunit B (PDGF subunit | Target 96 Cardiovascular II |
| B) | panel, Olink |
| Poly [ADP-ribose] polymerase 1 (PARP-1) | Target 96 Cardiovascular II |
| | panel, Olink |
| Polymeric immunoglobulin receptor (PIgR) | Target 96 Cardiovascular II |
| | panel, Olink |
| Programmed cell death 1 ligand 2 (PD-L2) | Target 96 Cardiovascular II |
| | panel, Olink |
| Proheparin-binding EGF-like growth factor (HB-EGF) | Target 96 Cardiovascular II |
| | panel, Olink |
| Pro-interleukin-16 (IL16) | Target 96 Cardiovascular II |
| | panel, Olink |
| Prolargin (PRELP) | Target 96 Cardiovascular II |
| | panel, Olink |
| Prostasin (PRSS8) | Target 96 Cardiovascular II |
| | panel, Olink |
| Protein AMBP (AMBP) | Target 96 Cardiovascular II |
| | panel, Olink |
| Proteinase-activated receptor 1 (PAR-1) | Target 96 Cardiovascular II |
| | panel, Olink |
| Protein-glutamine gamma-glutamyltransferase 2 | Target 96 Cardiovascular II |
| (TGM2) | panel, Olink |
| Proto-oncogene tyrosine-protein kinase Src (SRC) | Target 96 Cardiovascular II |
| | panel, Olink |
| P-selectin glycoprotein ligand 1 (PSGL-1) | Target 96 Cardiovascular II |
| | panel, Olink |
| Receptor for advanced glycosylation end products | Target 96 Cardiovascular II |
| (RAGE) | panel, Olink |
| Renin (REN) | Target 96 Cardiovascular II |
| | panel, Olink |
| Serine protease 27 (PRSS27) | Target 96 Cardiovascular II |
| | panel, Olink |
| Serine/threonine-protein kinase 4 (STK4) | Target 96 Cardiovascular II |
| | panel, Olink |
| Serpin A12 (SERPINA12) | Target 96 Cardiovascular II |
| | panel, Olink |
| SLAM family member 5 (CD84) | Target 96 Cardiovascular II |
| | panel, Olink |
| SLAM family member 7 (SLAMF7) | Target 96 Cardiovascular II |
| | panel, Olink |
| Sortilin (SORT1) | Target 96 Cardiovascular II |
| | panel, Olink |
| Spondin-2 (SPON2) | Target 96 Cardiovascular II |
| | panel, Olink |
| Stem cell factor (SCF) | Target 96 Cardiovascular II |
| | panel, Olink |
| Superoxide dismutase [Mn], mitochondrial (SOD2) | Target 96 Cardiovascular II |
| | panel, Olink |
| T-cell surface glycoprotein CD4 (CD4) | Target 96 Cardiovascular II |
| | panel, Olink |
| Thrombomodulin TM | Target 96 Cardiovascular II |
| | panel, Olink |
| Thrombopoietin (THPO) | Target 96 Cardiovascular II |
| | panel, Olink |
| Thrombospondin-2 (THBS2) | Target 96 Cardiovascular II |
| | panel, Olink |
| Tissue factor (TF) | Target 96 Cardiovascular II |
| | panel, Olink |
| TNF-related apoptosis-inducing ligand receptor 2 | Target 96 Cardiovascular II |
| (TRAIL-R2) | panel, Olink |
| Tumor necrosis factor receptor superfamily member | Target 96 Cardiovascular II |
| 10A (TNFRSF10A) | panel, Olink |
| Tumor necrosis factor receptor superfamily member | Target 96 Cardiovascular II |
| 11A (TNFRSF11A) | panel, Olink |
| Tumor necrosis factor receptor superfamily member | Target 96 Cardiovascular II |
| 13B (TNFRSF13B) | panel, Olink |
| Tyrosine-protein kinase Mer (MERTK) | Target 96 Cardiovascular II |
| | panel, Olink |
| Vascular endothelial growth factor D (VEGFD) | Target 96 Cardiovascular II |
| | panel, Olink |
| V-set and immunoglobulin domain-containing protein 2 | Target 96 Cardiovascular II |
| (VSIG2) | panel, Olink |
| Aminopeptidase N (AP-N) | Target 96 Cardiovascular III |
| | panel, Olink |
| Azurocidin (AZU1) | Target 96 Cardiovascular III |
| | panel, Olink |
| Bleomycin hydrolase (BLM hydrolase) | Target 96 Cardiovascular III |
| | panel, Olink |
| Cadherin-5 (CDH5) | Target 96 Cardiovascular III |
| | panel, Olink |
| Carboxypeptidase A1 (CPA1) | Target 96 Cardiovascular III |
| | panel, Olink |
| Carboxypeptidase B (CPB1) | Target 96 Cardiovascular III |
| | panel, Olink |
| Caspase-3 (CASP-3) | Target 96 Cardiovascular III |
| | panel, Olink |
| Cathepsin D (CTSD) | Target 96 Cardiovascular III |
| | panel, Olink |
| Cathepsin Z (CTSZ) | Target 96 Cardiovascular III |
| | panel, Olink |
| C-C motif chemokine 15 (CCL15) | Target 96 Cardiovascular III |
| | panel, Olink |
| C-C motif chemokine 16 (CCL16) | Target 96 Cardiovascular III |
| | panel, Olink |
| C-C motif chemokine 24 (CCL24) | Target 96 Cardiovascular III |
| | panel, Olink |
| CD166 antigen (ALCAM) | Target 96 Cardiovascular III |
| | panel, Olink |
| Chitinase-3-like protein 1 (CHI3L1) | Target 96 Cardiovascular III |
| | panel, Olink |
| Chitotriosidase-1 (CHIT1) | Target 96 Cardiovascular III |
| | panel, Olink |
| Collagen alpha-1(I) chain (COL1A1) | Target 96 Cardiovascular III |
| | panel, Olink |
| Complement component C1q receptor (CD93) | Target 96 Cardiovascular III |
| | panel, Olink |
| Contactin-1 (CNTN1) | Target 96 Cardiovascular III |
| | panel, Olink |
| C-X-C motif chemokine 16 (CXCL16) | Target 96 Cardiovascular III |
| | panel, Olink |
| Cystatin-B (CSTB) | Target 96 Cardiovascular III |
| | panel, Olink |
| Elafin (PI3) | Target 96 Cardiovascular III |
| | panel, Olink |
| Ephrin type-B receptor 4 (EPHB4) | Target 96 Cardiovascular III |
| | panel, Olink |
| Epidermal growth factor receptor (EGFR) | Target 96 Cardiovascular III |
| | panel, Olink |
| Epithelial cell adhesion molecule (Ep-CAM) | Target 96 Cardiovascular III |
| | panel, Olink |
| E-selectin (SELE) | Target 96 Cardiovascular III |
| | panel, Olink |
| Fatty acid-binding protein, adipocyte (FABP4) | Target 96 Cardiovascular III |
| | panel, Olink |
| Galectin-3 (Gal-3) | Target 96 Cardiovascular III |
| | panel, Olink |
| Galectin-4 (Gal-4) | Target 96 Cardiovascular III |
| | panel, Olink |
| Granulins (GRN) | Target 96 Cardiovascular III |
| | panel, Olink |
| Growth/differentiation factor 15 (GDF-15) | Target 96 Cardiovascular III |
| | panel, Olink |
| Insulin-like growth factor-binding protein 1 (IGFBP-1) | Target 96 Cardiovascular III |
| | panel, Olink |
| Insulin-like growth factor-binding protein 2 (IGFBP-2) | Target 96 Cardiovascular III |
| | panel, Olink |
| Insulin-like growth factor-binding protein 7 (IGFBP-7) | Target 96 Cardiovascular III |
| | panel, Olink |
| Integrin beta-2 (ITGB2) | Target 96 Cardiovascular III |
| | panel, Olink |
| Intercellular adhesion molecule 2 (ICAM-2) | Target 96 Cardiovascular III |
| | panel, Olink |
| Interleukin-1 receptor type 1 (IL-1RT1) | Target 96 Cardiovascular III |
| | panel, Olink |
| Interleukin-1 receptor type 2 (IL-1RT2) | Target 96 Cardiovascular III |
| | panel, Olink |
| Interleukin-2 receptor subunit alpha (IL2-RA) | Target 96 Cardiovascular III |
| | panel, Olink |
| Interleukin-6 receptor subunit alpha (IL-6RA) | Target 96 Cardiovascular III |
| | panel, Olink |
| Interleukin-17 receptor A (IL-17RA) | Target 96 Cardiovascular III |
| | panel, Olink |
| Interleukin-18-binding protein (IL-18BP) | Target 96 Cardiovascular III |
| | panel, Olink |
| Junctional adhesion molecule A (JAM-A) | Target 96 Cardiovascular III |
| | panel, Olink |
| Kallikrein-6 (KLK6) | Target 96 Cardiovascular III |
| | panel, Olink |
| Low-density lipoprotein receptor (LDL receptor) | Target 96 Cardiovascular III |
| | panel, Olink |
| Lymphotoxin-beta receptor (LTBR) | Target 96 Cardiovascular III |
| | panel, Olink |
| Matrix extracellular phosphoglycoprotein (MEPE) | Target 96 Cardiovascular III |
| | panel, Olink |
| Matrix metalloproteinase-2 (MMP-2) | Target 96 Cardiovascular III |
| | panel, Olink |
| Matrix metalloproteinase-3 (MMP-3) | Target 96 Cardiovascular III |
| | panel, Olink |
| Matrix metalloproteinase-9 (MMP-9) | Target 96 Cardiovascular III |
| | panel, Olink |
| Metalloproteinase inhibitor 4 (TIMP4) | Target 96 Cardiovascular III |
| | panel, Olink |
| Monocyte chemotactic protein 1 (MCP-1) | Target 96 Cardiovascular III |
| | panel, Olink |
| Myeloblastin (PRTN3) | Target 96 Cardiovascular III |
| | panel, Olink |
| Myeloperoxidase (MPO) | Target 96 Cardiovascular III |
| | panel, Olink |
| Myoglobin (MB) | Target 96 Cardiovascular III |
| | panel, Olink |
| Neurogenic locus notch homolog protein 3 (Notch 3) | Target 96 Cardiovascular III |
| | panel, Olink |
| N-terminal prohormone brain natriuretic peptide (NT- | Target 96 Cardiovascular III |
| proBNP) | panel, Olink |
| Osteopontin (OPN) | Target 96 Cardiovascular III |
| | panel, Olink |
| Osteoprotegerin (OPG) | Target 96 Cardiovascular III |
| | panel, Olink |
| Paraoxonase (PON3) | Target 96 Cardiovascular III |
| | panel, Olink |
| Peptidoglycan recognition protein 1 (PGLYRP1) | Target 96 Cardiovascular III |
| | panel, Olink |
| Perlecan (PLC) | Target 96 Cardiovascular III |
| | panel, Olink |
| Plasminogen activator inhibitor 1 (PAI) | Target 96 Cardiovascular III |
| | panel, Olink |
| Platelet endothelial cell adhesion molecule (PECAM-1) | Target 96 Cardiovascular III |
| | panel, Olink |
| Platelet-derived growth factor subunit A (PDGF subunit | Target 96 Cardiovascular III |
| A) | panel, Olink |
| Platelet glycoprotein VI (GP6) | Target 96 Cardiovascular III |
| | panel, Olink |
| Proprotein convertase subtilisin/kexin type 9 (PCSK9) | Target 96 Cardiovascular III |
| | panel, Olink |
| Protein delta homolog 1 (DLK-1) | Target 96 Cardiovascular III |
| | panel, Olink |
| P-selectin (SELP) | Target 96 Cardiovascular III |
| | panel, Olink |
| Pulmonary surfactant-associated protein D (PSP-D) | Target 96 Cardiovascular III |
| | panel, Olink |
| Resistin (RETN) | Target 96 Cardiovascular III |
| | panel, Olink |
| Retinoic acid receptor responder protein 2 (RARRES2) | Target 96 Cardiovascular III |
| | panel, Olink |
| Scavenger receptor cysteine-rich type 1 protein M130 | Target 96 Cardiovascular III |
| (CD163) | panel, Olink |
| Secretoglobin family 3A member 2 (SCGB3A2) | Target 96 Cardiovascular III |
| | panel, Olink |
| Spondin-1 (SPON1) Q9HCB6 | Target 96 Cardiovascular III |
| | panel, Olink |
| ST2 protein (ST2) Q01638 | Target 96 Cardiovascular III |
| | panel, Olink |
| Tartrate-resistant acid phosphatase type 5 (TR-AP) | Target 96 Cardiovascular III |
| P13686 | panel, Olink |
| Tissue factor pathway inhibitor (TFPI) P10646 | Target 96 Cardiovascular III |
| | panel, Olink |
| Tissue-type plasminogen activator (t-PA) P00750 | Target 96 Cardiovascular III |
| | panel, Olink |
| Transferrin receptor protein 1 (TR) P02786 | Target 96 Cardiovascular III |
| | panel, Olink |
| Trefoil factor 3 (TFF3) Q07654 | Target 96 Cardiovascular III |
| | panel, Olink |
| Trem-like transcript 2 protein (TLT-2) Q5T2D2 | Target 96 Cardiovascular III |
| | panel, Olink |
| Tumor necrosis factor ligand superfamily member 13B | Target 96 Cardiovascular III |
| (TNFSF13B) | panel, Olink |
| Tumor necrosis factor receptor 1 (TNF-R1) | Target 96 Cardiovascular III |
| | panel, Olink |
| Tumor necrosis factor receptor 2 (TNF-R2) | Target 96 Cardiovascular III |
| | panel, Olink |
| Tumor necrosis factor receptor superfamily member 6 | Target 96 Cardiovascular III |
| (FAS) | panel, Olink |
| Tumor necrosis factor receptor superfamily member | Target 96 Cardiovascular III |
| 10C (TNFRSF10C) | panel, Olink |
| Tumor necrosis factor receptor superfamily member 14 | Target 96 Cardiovascular III |
| (TNFRSF14) | panel, Olink |
| Tyrosine-protein kinase receptor UFO (AXL) | Target 96 Cardiovascular III |
| | panel, Olink |
| Tyrosine-protein phosphatase non-receptor type | Target 96 Cardiovascular III |
| substrate 1 (SHPS-1) | panel, Olink |
| Urokinase plasminogen activator surface receptor (U- | Target 96 Cardiovascular III |
| PAR) | panel, Olink |
| Urokinase-type plasminogen activator (uPA) | Target 96 Cardiovascular III |
| | panel, Olink |
| von Willebrand factor (vWF) | Target 96 Cardiovascular III |
| | panel, Olink |
| Adenosine Deaminase (ADA) | Target 96 Inflammation |
| | panel, Olink |
| Artemin (ARTN) | Target 96 Inflammation |
| | panel, Olink |
| Axin-1 (AXIN1) | Target 96 Inflammation |
| | panel, Olink |
| Beta-nerve growth factor (Beta-NGF) | Target 96 Inflammation |
| | panel, Olink |
| Caspase-8 (CASP-8) | Target 96 Inflammation |
| | panel, Olink |
| C-C motif chemokine 3 (CCL3) | Target 96 Inflammation |
| | panel, Olink |
| C-C motif chemokine 4 (CCL4) | Target 96 Inflammation |
| | panel, Olink |
| C-C motif chemokine 19 (CCL19) | Target 96 Inflammation |
| | panel, Olink |
| C-C motif chemokine 20 (CCL20) | Target 96 Inflammation |
| | panel, Olink |
| C-C motif chemokine 23 (CCL23) | Target 96 Inflammation |
| | panel, Olink |
| C-C motif chemokine 25 (CCL25) | Target 96 Inflammation |
| | panel, Olink |
| C-C motif chemokine 28 (CCL28) | Target 96 Inflammation |
| | panel, Olink |
| CD40L receptor (CD40) | Target 96 Inflammation |
| | panel, Olink |
| CUB domain-containing protein 1 (CDCP1) | Target 96 Inflammation |
| | panel, Olink |
| C-X-C motif chemokine 1 (CXCL1) | Target 96 Inflammation |
| | panel, Olink |
| C-X-C motif chemokine 5 (CXCL5) | Target 96 Inflammation |
| | panel, Olink |
| C-X-C motif chemokine 6 (CXCL6) | Target 96 Inflammation |
| | panel, Olink |
| C-X-C motif chemokine 9 (CXCL9) | Target 96 Inflammation |
| | panel, Olink |
| C-X-C motif chemokine 10 (CXCL10) | Target 96 Inflammation |
| | panel, Olink |
| C-X-C motif chemokine 11 (CXCL11) | Target 96 Inflammation |
| | panel, Olink |
| Cystatin D (CST5) | Target 96 Inflammation |
| | panel, Olink |
| Delta and Notch-like epidermal growth factor-related | Target 96 Inflammation |
| receptor (DNER) | panel, Olink |
| Eotaxin (CCL11) | Target 96 Inflammation |
| | panel, Olink |
| Eukaryotic translation initiation factor 4E-binding | Target 96 Inflammation |
| protein 1 (4E-BP1) | panel, Olink |
| Fibroblast growth factor 21 (FGF-21) | Target 96 Inflammation |
| | panel, Olink |
| Fibroblast growth factor 23 (FGF-23) | Target 96 Inflammation |
| | panel, Olink |
| Fibroblast growth factor 5 (FGF-5) | Target 96 Inflammation |
| | panel, Olink |
| Fibroblast growth factor 19 (FGF-19) | Target 96 Inflammation |
| | panel, Olink |
| Fms-related tyrosine kinase 3 ligand (FIt3L) | Target 96 Inflammation |
| | panel, Olink |
| Fractalkine (CX3CL1) | Target 96 Inflammation |
| | panel, Olink |
| Glial cell line-derived neurotrophic factor (GDNF) | Target 96 Inflammation |
| | panel, Olink |
| Hepatocyte growth factor (HGF) | Target 96 Inflammation |
| | panel, Olink |
| Interferon gamma (IFN-gamma) | Target 96 Inflammation |
| | panel, Olink |
| Interleukin-1 alpha (IL-1 alpha) | Target 96 Inflammation |
| | panel, Olink |
| Interleukin-2 (IL-2) | Target 96 Inflammation |
| | panel, Olink |
| Interleukin-2 receptor subunit beta (IL-2RB) | Target 96 Inflammation |
| | panel, Olink |
| Interleukin-4 (IL-4) | Target 96 Inflammation |
| | panel, Olink |
| Interleukin-5 (IL5) | Target 96 Inflammation |
| | panel, Olink |
| Interleukin-6 (IL6) | Target 96 Inflammation |
| | panel, Olink |
| Interleukin-7 (IL-7) | Target 96 Inflammation |
| | panel, Olink |
| Interleukin-8 (IL-8) | Target 96 Inflammation |
| | panel, Olink |
| Interleukin-10 (IL10) | Target 96 Inflammation |
| | panel, Olink |
| Interleukin-10 receptor subunit alpha (IL-10RA) | Target 96 Inflammation |
| | panel, Olink |
| Interleukin-10 receptor subunit beta (IL-10RB) | Target 96 Inflammation |
| | panel, Olink |
| Interleukin-12 subunit beta (IL-12B) | Target 96 Inflammation |
| | panel, Olink |
| Interleukin-13 (IL-13) | Target 96 Inflammation |
| | panel, Olink |
| Interleukin-15 receptor subunit alpha (IL-15RA) | Target 96 Inflammation |
| | panel, Olink |
| Interleukin-17A (IL-17A) | Target 96 Inflammation |
| | panel, Olink |
| Interleukin-17C (IL-17C) | Target 96 Inflammation |
| | panel, Olink |
| Interleukin-18 (IL-18) | Target 96 Inflammation |
| | panel, Olink |
| Interleukin-18 receptor 1 (IL-18R1) | Target 96 Inflammation |
| | panel, Olink |
| Interleukin-20 (IL-20) | Target 96 Inflammation |
| | panel, Olink |
| Interleukin-20 receptor subunit alpha (IL-20RA) | Target 96 Inflammation |
| | panel, Olink |
| Interleukin-22 receptor subunit alpha-1 (IL-22 RA1) | Target 96 Inflammation |
| | panel, Olink |
| Interleukin-24 (IL-24) | Target 96 Inflammation |
| | panel, Olink |
| Interleukin-33 (IL-33) | Target 96 Inflammation |
| | panel, Olink |
| Latency-associated peptide transforming growth factor | Target 96 Inflammation |
| beta-1 (LAP TGF-beta-1) | panel, Olink |
| Leukemia inhibitory factor (LIF) | Target 96 Inflammation |
| | panel, Olink |
| Leukemia inhibitory factor receptor (LIF-R) | Target 96 Inflammation |
| | panel, Olink |
| Macrophage colony-stimulating factor 1 (CSF-1) | Target 96 Inflammation |
| | panel, Olink |
| Matrix metalloproteinase-1 (MMP-1) | Target 96 Inflammation |
| | panel, Olink |
| Matrix metalloproteinase-10 (MMP-10) | Target 96 Inflammation |
| | panel, Olink |
| Monocyte chemotactic protein 1 (MCP-1) | Target 96 Inflammation |
| | panel, Olink |
| Monocyte chemotactic protein 2 (MCP-2) | Target 96 Inflammation |
| | panel, Olink |
| Monocyte chemotactic protein 3 (MCP-3) | Target 96 Inflammation |
| | panel, Olink |
| Monocyte chemotactic protein 4 (MCP-4) | Target 96 Inflammation |
| | panel, Olink |
| Natural killer cell receptor 2B4 (CD244) | Target 96 Inflammation |
| | panel, Olink |
| Neurotrophin-3 (NT-3) | Target 96 Inflammation |
| | panel, Olink |
| Neurturin (NRTN) | Target 96 Inflammation |
| | panel, Olink |
| Oncostatin-M (OSM) | Target 96 Inflammation |
| | panel, Olink |
| Osteoprotegerin (OPG) | Target 96 Inflammation |
| | panel, Olink |
| Programmed cell death 1 ligand 1 (PD-L1) | Target 96 Inflammation |
| | panel, Olink |
| Protein S100-A12 (EN-RAGE) | Target 96 Inflammation |
| | panel, Olink |
| Signaling lymphocytic activation molecule (SLAMF1) | Target 96 Inflammation |
| | panel, Olink |
| SIR2-like protein 2 (SIRT2) | Target 96 Inflammation |
| | panel, Olink |
| STAM-binding protein (STAMBP) | Target 96 Inflammation |
| | panel, Olink |
| Stem cell factor (SCF) | Target 96 Inflammation |
| | panel, Olink |
| Sulfotransferase 1A1 (ST1A1) | Target 96 Inflammation |
| | panel, Olink |
| T cell surface glycoprotein CD6 isoform (CD6) | Target 96 Inflammation |
| | panel, Olink |
| T-cell surface glycoprotein CD5 (CD5) | Target 96 Inflammation |
| | panel, Olink |
| T-cell surface glycoprotein CD8 alpha chain (CD8A) | Target 96 Inflammation |
| | panel, Olink |
| Thymic stromal lymphopoietin (TSLP) | Target 96 Inflammation |
| | panel, Olink |
| TNF-beta (TNFB) | Target 96 Inflammation |
| | panel, Olink |
| TNF-related activation-induced cytokine (TRANCE) | Target 96 Inflammation |
| | panel, Olink |
| TNF-related apoptosis-inducing ligand (TRAIL) | Target 96 Inflammation |
| | panel, Olink |
| Transforming growth factor alpha (TGF-alpha) | Target 96 Inflammation |
| | panel, Olink |
| Tumor necrosis factor (Ligand) superfamily, member 12 | Target 96 Inflammation |
| (TWEAK) | panel, Olink |
| Tumor necrosis factor (TNF) | Target 96 Inflammation |
| | panel, Olink |
| Tumor necrosis factor ligand superfamily member 14 | Target 96 Inflammation |
| (TNFSF14) | panel, Olink |
| Tumor necrosis factor receptor superfamily member 9 | Target 96 Inflammation |
| (TNFRSF9) | panel, Olink |
| Urokinase-type plasminogen activator (uPA) | Target 96 Inflammation |
| | panel, Olink |
| Vascular endothelial growth factor A (VEGF-A) | Target 96 Inflammation |
| | panel, Olink |

We claim:

1. A method of predicting a response to a treatment regimen for psoriasis in a subject in need thereof, the method comprising:
a. obtaining a sample from the subject;
b. contacting the sample with a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A);
c. obtaining a panel of clinical variables from the subject comprising disease duration, body mass index (BMI), weight, age, sex, treatment history, Dermatology Life Quality Index (DLQI) score, and Psoriasis Area and Severity Index (PASI);
d. analyzing a range of threshold values of the panel of biomarkers and the panel of clinical variables to determine a predictive value for the subject, wherein a predictive value of less than about 0.1 indicates that the subject is less likely to respond to the treatment regimen than a subject with a predictive value greater than about 0.1;
e. determining the treatment duration for the subject with the treatment regimen based on the predictive value, with a score of greater than about 0.1 indicating treating the subject for a shorter duration and a score of less than about 0.1 indicating treating the subject for a longer duration; and
f. treating the subject with the treatment regimen for a duration based on the score.

2. The method of claim 1, wherein the contacting step comprises contacting the samples with an isolated set of probes corresponding to the panel of biomarkers.

3. The method of claim 2, wherein the sample is a blood sample.

4. The method of claim 1, wherein the method further comprises administering a therapeutic agent to the subject to treat or prevent the psoriasis.

5. The method of claim 4, wherein the therapeutic agent is an anti-IL-23 antibody.

6. The method of claim 5, wherein the anti-IL-23 antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3.

7. The method of claim 5, wherein the anti-IL-23 antibody comprises a light chain variable region amino acid sequence of SEQ ID NO: 8 and a heavy chain variable region amino acid sequence of SEQ ID NO: 7.

8. The method of claim 5, wherein the anti-IL-23 antibody comprises a light chain amino acid sequence of SEQ ID NO: 10 and a heavy chain amino acid sequence of SEQ ID NO: 9.

9. The method of claim 5, wherein the anti-IL-23 antibody is guselkumab.

10. The method of claim 6, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

11. The method of claim 1, wherein the analyzing step is performed using a machine learning module.

12. The method of claim 11, wherein the machine learning model comprises at least one of a support vector machine module, a random forest module, a logistic regression module, and a gradient tree boosting module.

13. The method of claim 1, wherein the shorter treatment duration is less than 68 weeks.

14. The method of claim 1, wherein the longer treatment duration is greater than 68 weeks.

15. The method of claim 6, wherein the sample and panel of clinical variables are obtained prior to the treatment regimen and/or at week 4, 12, 16, 20, 28, 36, 44, 52, 60, or 68 of treatment.

16. The method of claim 1, wherein the panel of clinical variables further comprises change in PASI.

17. A method of predicting a response to a treatment regimen with an anti-IL-23 antibody and treating for moderate to severe plaque psoriasis in a subject in need thereof, the method comprising:
a. obtaining a sample from the subject;
b. contacting the sample with a panel of biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A);
c. obtaining a panel of clinical variables from the subject comprising disease duration, body mass index (BMI), weight, age, sex, treatment history, Dermatology Life Quality Index (DLQI) score, and Psoriasis Area and Severity Index (PASI);
d. analyzing a range of threshold values of the panel of biomarkers and the panel of clinical variables to determine a predictive value for the subject, wherein a predictive value of less than about 0.1 indicates that the subject is less likely to be a super responder to the treatment regimen than a subject with a predictive value greater than about 0.1;
e. determining the treatment duration for the subject with the treatment regimen based on the predictive value, with a score of less than about 0.1 indicating that the subject will not be a super responder to treatment duration and a score of greater than about 0.1 indicating that the subject will be a super responder to the treatment regimen; and
f. treating the subject with the treatment regimen for a duration based on the score.

18. The method of claim 17, wherein the subject has a score of greater than zero, further comprising treating the subject with the anti-IL-23 antibody for a period of 68 weeks and ceasing treatment 68 weeks after initial treatment.

19. The method of claim 17, wherein the anti-IL-23 antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3.

20. The method of claim 19, wherein the anti-IL-23 antibody comprises a light chain variable region amino acid sequence of SEQ ID NO: 8 and a heavy chain variable region amino acid sequence of SEQ ID NO: 7.

21. The method of claim 20, wherein the anti-IL-23 antibody comprises a light chain amino acid sequence of SEQ ID NO: 10 and a heavy chain amino acid sequence of SEQ ID NO: 9.

22. The method of claim 21, wherein the anti-IL-23 antibody is guselkumab.

23. The method of claim 22, wherein the anti-IL-23 antibody is administered subcutaneously at a dose of 100 mg per administration.

24. The method of claim 23, wherein the antibody is administered in an initial dose, 4 weeks after the initial dose and every 8 weeks after the dose at 4 weeks.

25. The method of claim 24, wherein the antibody is administered every 8 or 16 weeks after a dose at 28 weeks.

26. The method of claim 19, wherein a predictive value of 0 indicates that the subject is less likely to respond to the treatment regimen than a subject with a predictive value of 1.

27. A kit for predicting a response to a treatment regimen for psoriasis in a subject in need thereof, the kit comprising:
a. an isolated set of probes capable of detecting a panel of biomarkers comprising at least one, such as 1, 2, 3, 4, 5, 6, 7, 8, 9, 10, 11, or more, biomarkers comprising fibroblast growth factor 19 (FGF19), scavenger receptor cysteine-rich type 1 protein M130 (CD163), integrin beta-2 (ITGB2), interleukin 17F (IL-17F), beta-defensin-2 (BD-2), suppression of tumorigenicity 2 protein (ST2), interleukin 22 (IL-22), interleukin 19 (IL-19), elafin/peptidase inhibitor 3 (PI3), interleukin-10 receptor subunit alpha (IL-10RA), and interleukin 17A (IL-17A); and
b. instructions for use.
